(19) United States
(12) Patent Application Publication
(10) Pub. No.: US 2024/0302375 A1
(43) Pub. Date: Sep. 12, 2024

(54) PAPER-BASED DEVICE AND USES THEREOF FOR MONITORING RECURRENCE OF CANCERS

(71) Applicant: VANDERBILT UNIVERSITY, Nashville, TN (US)

(72) Inventor: Emily C. KIGHT, Nashville, TN (US)

(21) Appl. No.: 18/689,241

(22) PCT Filed: Sep. 7, 2022
(86) PCT No.: PCT/US2022/042731

§ 371 Date: Mar. 5, 2024

Related U.S. Application Data
(60) Provisional application No. 63/241,292, filed on Sep. 7, 2021.

(51) Int. Cl. G01N 33/574; A61K 31/337; A61K 31/555; A61K 33/243; G01N 21/77; G01N 33/68; G01N 33/70; G16H 50/20

(52) U.S. Cl. CPC G01N 33/57449; A61K 31/337; A61K 31/555; A61K 33/243; G01N 21/77; G01N 33/6893; G01N 33/70; G16H 50/20; G01N 2021/7759

(57) ABSTRACT

The present disclosure relates to a device and uses thereof for detecting, treating, and monitoring cancers.

### CROSS REFERENCE TO RELATED APPLICATIONS

This application claims the priority benefit of U.S. Provisional Application No. 63/241,292, filed Sep. 7, 2021, which is expressly incorporated herein by reference in its entirety.

### FIELD

The present disclosure relates to a paper-based device and uses thereof for detecting, treating, and monitoring cancers.

### BACKGROUND

Despite treatment, up to 70% of patients with ovarian cancer (OC) develop recurrence of disease, and patients faced with a high relapse rate are concerned about recurrence. Even though OC patients undergo long-term surveillance to improve recurrence detection, current diagnostic methods, such as pelvic examination, transvaginal ultrasonography, and blood tests such as cancer antigen 125 (CA-125), are invasive and require a clinic visit. Factors such as making an appointment to have blood work done, traveling to the location, a lack of confidence in their symptoms (cramping, bloating, frequent urination), being too busy, not wanting to bother a doctor and the lack of frequency of testing were highlighted concerns of OC patients. Currently, there is no at-home monitoring test for ovarian cancer recurrence that can address many of these factors. As many cancer patients eventually relapse, there is a great need to develop at-home detection methods to monitor molecular biomarker levels indicative of recurrence and reduce mortality. What is needed are device and methods for monitoring cancers.

### SUMMARY

In some aspects, disclosed herein is a method of monitoring recurrence of a cancer in a subject, comprising
obtaining a biological sample from the subject's urine;
contacting the biological sample with a first test paper comprising a first reagent area that changes in color shade after contacting a human epididymis protein 4 (HE4) polypeptide and a second test paper comprising a second reagent area that changes in color shade after contacting a creatinine (CRE) polypeptide;
scanning the first test paper and second test paper to determine the intensity of the color shade of the first reagent area and the intensity of the color shade of the second reagent area;
calculating the ratio of the intensity of the color shade of the first reagent area and the intensity of the color shade of the second reagent area, and
determining that the subject has recurrence of the cancer if the ratio is greater than about 2.

In some embodiments, the test paper is scanned by a cell phone or a scanner. In some embodiments, the cancer is ovarian cancer.

In some embodiments, the method further comprises administering to the subject a therapeutically effective amount of a cancer therapeutic agent if the subject has recurrence of the cancer. In some embodiments, the cancer therapeutic agent is selected from the group consisting of cisplatin, carboplatin, paclitaxel, and docetaxel.

In some aspects, disclosed herein is a method of treating a cancer in a subject in need thereof, comprising
obtaining a biological sample from the subject's urine;
contacting the biological sample with a first test paper comprising a first reagent area that changes in color shade after contacting a human epididymis protein 4 (HE4) polypeptide and a second test paper comprising a second reagent area that changes in color shade after contacting a creatinine (CRE) polypeptide;
scanning the first test paper and second test paper to determine the intensity of the color shade of the first reagent area and the intensity of the color shade of the second reagent area;
calculating the ratio of the intensity of the color shade of the first reagent area and the intensity of the color shade of the second reagent area;
determining that the subject has recurrence of the cancer if the ratio is greater than about 2; and
administering to the subject a therapeutically effective amount of a cancer therapeutic agent if the subject has recurrence of the cancer.

Also disclosed herein is a method of monitoring recurrence of a cancer in a subject, comprising
a) obtaining a first biological sample from the subject's urine immediately after a cancer therapy;
b) obtaining a second biological sample from the subject's urine after step a);
c) contacting the first biological sample and the second biological sample with a first test paper, wherein the first test paper comprises a first reagent area that changes in color shade after contacting a human epididymis protein 4 (HE4) polypeptide and a second test paper comprising a second reagent area that changes in color shade after contacting a creatinine (CRE) polypeptide;
d) scanning the first test paper and second test paper to determine the intensity of the color shade of the first reagent area and the intensity of the color shade of the second reagent area;
e) calculating the ratio of the intensity of the color shade of the first reagent area and the intensity of the color shade of the second reagent area for the first biological sample and the second biological sample respectively, and
f) determining that the subject has recurrence of the cancer if the ratio of the second biological sample is greater than the ratio of the first biological sample.

In some embodiments, the test paper is scanned by a cell phone or a scanner. In some embodiments, the cancer is ovarian cancer. In some embodiments, the cancer therapy is a surgery or a chemotherapy treatment. In some embodiments, the cancer therapy partially or fully eliminated the cancer in the subject. In some embodiments, the method disclosed herein further comprises administering to the subject a therapeutically effective amount of a cancer therapeutic agent if the subject has recurrence of the cancer.

Also disclosed herein is a method of determining a subject's response to a cancer therapy, comprising
obtaining a first biological sample from the subject's urine prior to the cancer therapy;
obtaining a second biological sample from the subject's urine after the cancer therapy;
contacting the first biological sample and the second biological sample with a first test paper, wherein the first test paper comprises a first reagent area that changes in color shade after contacting a human epididymis protein 4 (HE4) polypeptide and a second test paper comprising a second reagent area that changes in color shade after contacting a creatinine (CRE) polypeptide;
scanning the first test paper and the second test paper to determine the intensity of the color shade of the first reagent area and the intensity of the color shade of the second reagent area;
calculating the ratio of the intensity of the color shade of the first reagent area and the intensity of the color shade of the second reagent area for the first biological sample and the second biological sample respectively, and
determining:
   the subject as having resistance to the cancer therapy if the ratio of the second biological sample is equal or greater than the ratio of the first biological sample; or
   the subject as responding to the cancer therapy if the ratio of the second biological sample is less than the ratio of the first biological sample.

### BRIEF DESCRIPTION OF THE DRAWINGS

The accompanying figures, which are incorporated in and constitute a part of this specification, illustrate several aspects described below.

FIG. 1 shows a comparison between flatbed scanner and cell phone measurement of HE4 pM concentrations (top) and comparison between flatbed scanner and cell phone measurement of creatinine (bottom). Error bars the percent error found for each concentration (Mean+s.d, n=2).

FIG. 2 shows simulated clinical patient ratios of urinary HE4/CRE. Each image shows a lateral flow strip and corresponding creatinine strip below. In the LFA strip, the left line is the test line and the right line is the control line. * LMP=low malignancy potential cyst. The labels below are the ratio of HE4/CRE and the corresponding stage of ovarian cancer stage given Liao et al (2015) and Hellstrom et al. (2010).

FIG. 3 shows comparison of measured HE4/CRE ratios for scanner and cellphone to actual values (n=1).

FIG. 4 shows schematic of the two paper test strips and components. For the HE4 test strip (top), the sandwich assay captures the biomarker in the middle at the test line. The control line is generated when excess reporter binds. The intensity of the purple color (bottom) indicates the amount of creatinine. The two strips are placed in a scanner or cassette for the cell phone to be analyzed.

FIG. 5 shows HE4 test line intensity as a function of the assay read time (Mean+s.d., n=2) (top panel). The color intensity is shown as a function of assay read time for three creatinine concentrations of 5 mg/dL, 20 mg/dL, and 40 mg/dL (Mean+s.d, n=3) (bottom panel).

FIGS. 6A-6B show calibration plots generated from the scanner. FIG. 6A shows standard curve for concentrations of HE4 (pM) (Mean+s.d, n=2). FIG. 6B shows standard curve for concentrations of creatinine.

FIGS. 7A-7C show overview of the smartphone quantification system. FIG. 7A shows image of the components (lens, fiber optics, cell phone attachments and strip cassette). FIG. 7B shows a CAD model providing an overview of the components used in developing the system. FIG. 7C shows schematic of the test-strip holder indicating the color detection scheme and region of interest (ROI) for HE4 and CRE.

FIGS. 8A-8B show calibration plots generated from the cell phone-based device. FIG. 8A shows standard curve for concentrations of HE4 (pM) (Mean+s.d, n=2). FIG. 8B shows standard curve for concentrations of creatinine (mg/dL) from cell phone (Mean+s.d, n=3).

FIG. 9 shows that dot blot test was conducted with three different antibody and three different membranes. For the three antibodies tested, MDI-10 showed the least non-specific binding for the control spots and strong signal for the positive spots.

FIG. 10 shows details of the optical fiber-based illumination scheme used to provide uniform illumination over the field of view of the camera.

FIGS. 11A-11I show workflow of the cell phone app.

FIGS. 12A-12B show results of a test with patient positive for ovarian cancer and a healthy urine sample. A difference at the 1:100 dilution level on the test strips was observed to differentiate the positive cancer sample from the negative healthy urine sample.

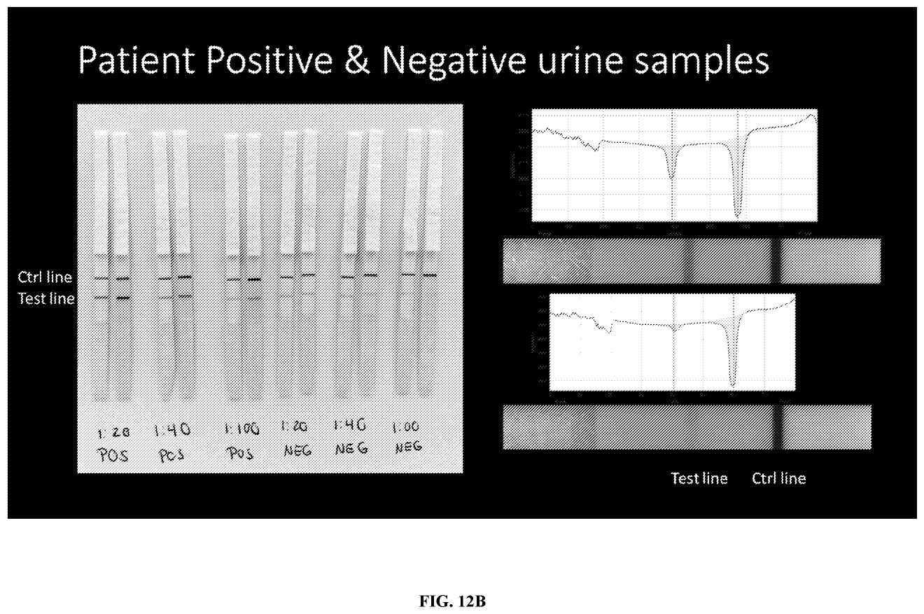

### DETAILED DESCRIPTION

Disclosed herein are compositions and methods which regulate the immune system for treating cancers and other immune disorders.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, urine, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used. The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs. In some embodiments herein, the biological sample is a urine sample.

The term “cancer” as used herein is defined as disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

A “control” is an alternative subject or sample used in an experiment for comparison purposes. A control can be “positive” or “negative.”

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “Recurrence-Free Interval (RFI)” is used herein to refer to time in years to first cancer recurrence censoring for second primary cancer or death without evidence of recurrence.

The term “Overall Survival (OS)” is used herein to refer to time in years from a treatment or a surgery to death from any cause.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. Administration of the therapeutic agents can be carried out at dosages and for periods of time effective for treatment of a subject.

“Therapeutically effective amount” or “therapeutically effective dose” of a composition refers to an amount that is effective to achieve a desired therapeutic result. In some embodiments, a desired therapeutic result is the treatment of a cancer. In some embodiments, a desired therapeutic result is reduction of tumor size. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

As used herein, the term “preventing” a disease, a disorder, or unwanted physiological event in a subject refers to the prevention of a disease, a disorder, or unwanted physiological event or prevention of a symptom of a disease, a disorder, or unwanted physiological event

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the term “therapeutic agent” is used, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The term “polypeptide” refers to a compound made up of a single chain of D- or L-amino acids or a mixture of D- and L-amino acids joined by peptide bonds.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “polynucleotide” refers to a single or double stranded polymer composed of nucleotide monomers.

### Devices and Methods

In some aspects, disclosed herein is a method of monitoring recurrence of a cancer in a subject, comprising
obtaining a biological sample from the subject's body fluid;
contact the biological sample with a test paper comprising a first reagent area that changes in color shade after contacting a human epididymis protein 4 (HE4) polypeptide and a second reagent area that changes in color shade after contacting a creatinine (CRE) polypeptide;
scanning the test paper to determine the intensity of the color shade of the first reagent area and the second reagent area;
calculating the ratio of the intensity of the color shade of the first reagent area and the intensity of the color shade of the second reagent area, and
determining that the subject has recurrence of the cancer if the ratio is greater than 1.

In some embodiments, the subject is determined as having recurrence of the cancer if the ratio is greater than 1 (for example, greater than 1.5, 2, 2.5, 3, 3.5, 4, 4.5, 5, 6, 7, 9, 10, 15, 20, 25, 30, 35, 50, 100, 1000, or 10,000).

In some aspects, disclosed herein is a method of treating a cancer in a subject in need thereof, comprising
obtaining a biological sample from the subject's urine;
contacting the biological sample with a first test paper comprising a first reagent area that changes in color shade after contacting a human epididymis protein 4 (HE4) polypeptide and a second test paper comprising a second reagent area that changes in color shade after contacting a creatinine (CRE) polypeptide;
scanning the first test paper and second test paper to determine the intensity of the color shade of the first reagent area and the intensity of the color shade of the second reagent area;
calculating the ratio of the intensity of the color shade of the first reagent area and the intensity of the color shade of the second reagent area;
determining that the subject has recurrence of the cancer if the ratio is greater than about 1; and
administering to the subject a therapeutically effective amount of a cancer therapeutic agent if the subject has recurrence of the cancer.

In some embodiments, the subject is determined as having recurrence of the cancer if the ratio is greater than 1 (for example, greater than 1.5, 2, 2.5, 3, 3.5, 4, 4.5, 5, 6, 7, 9, 10, 15, 20, 25, 30, 35, 50, 100, 1000, or 10,000).

In some embodiments, the first reagent area and the second reagent area are on one test paper. In some embodiments, the first reagent area and the second reagent area are on two different test papers.

In some embodiments, the body fluid sample, including, for example, a urine sample, a blood sample, a nasal swab sample, or an airway fluid sample.

In some embodiments, the method described herein can be used for monitoring a cancer that is selected from, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas, leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

In some embodiments, the body fluid sample is menstrual blood. Accordingly, in some embodiments, the method described herein is for monitoring recurrence of endometrial cancer.

In some embodiments, the body fluid sample is an airway fluid sample. Accordingly, in some embodiments, the method described herein is for monitoring recurrence of lung cancer.

In some embodiments, the body fluid sample is a urine sample. Accordingly, in some embodiments, the method described herein is for monitoring recurrence of ovarian cancer.

Accordingly, in some aspects, disclosed herein is a method of monitoring recurrence of ovarian cancer in a subject, comprising
obtaining a biological sample from the subject's urine;
contacting the biological sample with a first test paper comprising a first reagent area that changes in color shade after contacting a human epididymis protein 4 (HE4) polypeptide and a second test paper comprising a second reagent area that changes in color shade after contacting a creatinine (CRE) polypeptide;
scanning the first test paper and second test paper to determine the intensity of the color shade of the first reagent area and the intensity of the color shade of the second reagent area;
calculating the ratio of the intensity of the color shade of the first reagent area and the intensity of the color shade of the second reagent area, and
determining that the subject has recurrence of the cancer if the ratio is greater than about 1.

In some embodiments, the subject is determined as having recurrence of the cancer if the ratio is greater than 1 (for example, greater than 1.5, 2, 2.5, 3, 3.5, 4, 4.5, 5, 6, 7, 9, 10, 15, 20, 25, 30, 35, 50, 100, 1000, or 10,000).

In some embodiments, the test paper is scanned by a cell phone or a scanner. In some embodiments, the test paper is scanned by a cell phone.

In some embodiments, the method comprises a step of determining that the subject has recurrence of the cancer if the ratio is greater than about 2, greater than about 5, greater than about 10, greater than about 15, greater than about 20, greater than about 25, greater than about 30, greater than about 35, greater than about 40, greater than about 50, greater than about 60, greater than about 70, greater than about 80, greater than about 90, greater than about 100, greater than about 500, or greater than about 1000. In some embodiments, the ratio is from about 2 to about 47.

“Human epididymis protein 4” or “HE4” refers herein to a polypeptide that, in humans, is encoded by the WFDC2 gene. In some embodiments, the HE4 polypeptide is that identified in one or more publicly available databases as follows: HGNC: 15939, NCBI Entrez Gene: 10406, Ensembl: ENSG00000101443, OMIM®: 617548, UniProtKB/Swiss-Prot: Q14508.

Creatinine is a breakdown product of creatine phosphate from muscle and protein metabolism.

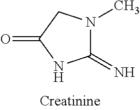

In some embodiments, the subject has cancer. In some embodiments, the subject has already been treated with a cancer therapy. In some embodiments, the cancer therapy and is surgery or a chemotherapy. In some embodiments, the cancer therapy partially or fully eliminated the cancer.

In some embodiments, the method further comprises administering to the subject a therapeutically effective amount of a cancer therapeutic agent if the subject has recurrence of the cancer. In some embodiments, the cancer therapeutic agent is selected from the group consisting of cisplatin, carboplatin, paclitaxel, and docetaxel.

Also disclosed herein is a method of monitoring recurrence of a cancer in a subject, comprising
a) obtaining a first biological sample from the subject immediately after a cancer therapy;
b) obtaining a second biological sample from the subject after step a);
c) contacting the first biological sample and the second biological sample with a test paper, wherein the test paper comprises a first reagent area that changes in color shade after contacting a human epididymis protein 4 (HE4) polypeptide or a second reagent area that changes in color shade after contacting a creatinine (CRE) polypeptide;
d) scanning the test paper to determine the intensity of the color shade of the first reagent area and the intensity of the color shade of the second reagent area;
e) calculating the ratio of the intensity of the color shade of the first reagent area and the intensity of the color shade of the second reagent area for the first biological sample and the second biological sample respectively, and
f) determining that the subject has recurrence of the cancer if the ratio of the second biological sample is greater than the ratio of the first biological sample.

The term “increased”, “increase”, or “greater” as used herein generally means an increase by a statically significant amount. In some embodiments, step f) comprises determining that the subject has recurrence of the cancer if the ratio of the second biological sample is about at least 10% greater than the ratio of the first biological sample (for example, at least about 20%, or at least about 30%, or at least about 40%, or at least about 50%, or at least about 60%, or at least about 70%, or at least about 80%, or at least about 90% or up to and including a 100% greater, or at least about a 2-fold, or at least about a 3-fold, or at least about a 4-fold, or at least about a 5-fold, or at least about a 10-fold, 100-fold, or at least 1000-fold greater as compared to the ratio of the first biological sample).

In some embodiments, the first reagent area and the second reagent area are on one test paper. In some embodiments, the first reagent area and the second reagent area are on two different test papers.

In some embodiments, the body fluid sample, including, for example, a urine sample, a blood sample, a nasal swab sample, or an airway fluid sample.

In some embodiments, the body fluid sample is menstrual blood. Accordingly, in some embodiments, the method described herein is for monitoring recurrence of endometrial cancer.

In some embodiments, the body fluid sample is an airway fluid sample. Accordingly, in some embodiments, the method described herein is for monitoring recurrence of lung cancer.

In some embodiments, the body fluid sample is a urine sample. Accordingly, in some embodiments, the method described herein is for monitoring recurrence of ovarian cancer.

Accordingly, disclosed herein is a method of monitoring recurrence of ovarian cancer in a subject, comprising
a) obtaining a first biological sample from the subject's urine immediately after a cancer therapy;
b) obtaining a second biological sample from the subject's urine after step a);
c) contacting the first biological sample and the second biological sample with a first test paper, wherein the first test paper comprises a first reagent area that changes in color shade after contacting a human epididymis protein 4 (HE4) polypeptide and a second test paper comprising a second reagent area that changes in color shade after contacting a creatinine (CRE) polypeptide;
d) scanning the first test paper and second test paper to determine the intensity of the color shade of the first reagent area and the intensity of the color shade of the second reagent area;
e) calculating the ratio of the intensity of the color shade of the first reagent area and the intensity of the color shade of the second reagent area for the first biological sample and the second biological sample respectively, and
f) determining that the subject has recurrence of the cancer if the ratio of the second biological sample is greater than the ratio of the first biological sample.

In some embodiments, step f) comprises determining that the subject has recurrence of the cancer if the ratio of the second biological sample is about at least 10% greater than the ratio of the first biological sample (for example, at least about 20%, or at least about 30%, or at least about 40%, or at least about 50%, or at least about 60%, or at least about 70%, or at least about 80%, or at least about 90% or up to and including a 100% greater, or at least about a 2-fold, or at least about a 3-fold, or at least about a 4-fold, or at least about a 5-fold, or at least about a 10-fold, 100-fold, or at least 1000-fold greater as compared to the ratio of the first biological sample).

The term “immediately after a cancer therapy” can be, for example, less than 1 minute after a cancer therapy, about 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days after a cancer therapy.

In some embodiments, the method disclosed herein further comprises obtaining a biological sample from the subject's urine prior to the cancer therapy (less than 1 minute prior to a cancer therapy, about 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days prior to the cancer therapy). In some embodiments, the method disclosed herein further comprises obtaining a biological sample from the subject's urine concurrently with the cancer therapy.

In some embodiments, the test paper is scanned by a cell phone or a scanner. In some embodiments, the cancer is ovarian cancer. In some embodiments, the cancer therapy is a surgery or a chemotherapy treatment. In some embodiments, the cancer therapy partially or fully eliminated the cancer in the subject. In some embodiments, the method disclosed herein further comprises administering to the subject a therapeutically effective amount of a cancer therapeutic agent if the subject has recurrence of the cancer.

Also, disclosed herein is a method of determining if a subject responds to a cancer therapy, comprising
obtaining a first biological sample from the subject prior to the cancer therapy;
obtaining a second biological sample from the subject after the cancer therapy;
contacting the first biological sample and the second biological sample with a test paper, wherein the test paper comprises a first reagent area that changes in color shade after contacting a human epididymis protein 4 (HE4) polypeptide or a second reagent area that changes in color shade after contacting a creatinine (CRE) polypeptide;
scanning the test paper to determine the intensity of the color shade of the first reagent area and the intensity of the color shade of the second reagent area;
calculating the ratio of the intensity of the color shade of the first reagent area and the intensity of the color shade of the second reagent area for the first biological sample and the second biological sample respectively, and
determining
the subject as having resistance to the cancer therapy if the ratio of the second biological sample is equal or greater than the ratio of the first biological sample; or
the subject as responding to the cancer therapy if the ratio of the second biological sample is less than the ratio of the first biological sample.

In some embodiments, the first reagent area and the second reagent area are on one test paper. In some embodiments, the first reagent area and the second reagent area are on two different test papers.

In some embodiments, the body fluid sample, including, for example, a urine sample, a blood sample, a nasal swab sample, or an airway fluid sample.

In some embodiments, the body fluid sample is menstrual blood. Accordingly, in some embodiments, the method described herein is for monitoring recurrence of endometrial cancer.

In some embodiments, the body fluid sample is an airway fluid sample. Accordingly, in some embodiments, the method described herein is for monitoring recurrence of lung cancer.

In some embodiments, the body fluid sample is a urine sample. Accordingly, in some embodiments, the method described herein is for monitoring recurrence of ovarian cancer.

Accordingly, also disclosed herein is a method of determining a subject's response to a cancer therapy, comprising
obtaining a first biological sample from the subject's urine prior to the cancer therapy;
obtaining a second biological sample from the subject's urine after the cancer therapy;
contacting the first biological sample and the second biological sample with a first test paper, wherein the first test paper comprises a first reagent area that changes in color shade after contacting a human epididymis protein 4 (HE4) polypeptide and a second test paper comprising a second reagent area that changes in color shade after contacting a creatinine (CRE) polypeptide;
scanning the first test paper and the second test paper to determine the intensity of the color shade of the first reagent area and the intensity of the color shade of the second reagent area;
calculating the ratio of the intensity of the color shade of the first reagent area and the intensity of the color shade of the second reagent area for the first biological sample and the second biological sample respectively, and
determining:
   the subject as having resistance to the cancer therapy if the ratio of the second biological sample is equal or greater than the ratio of the first biological sample; or
   the subject as responding to the cancer therapy if the ratio of the second biological sample is less than the ratio of the first biological sample.

In some embodiments, the test paper is scanned by a cell phone or a scanner. In some embodiments, the cancer is ovarian cancer. In some embodiments, the cancer therapy is a surgery or a chemotherapy treatment. In some embodiments, the cancer therapy is platinum.

Accordingly, the subject can be determined as platinum resistant if the ratio of the second biological sample is equal or greater than the ratio of the first biological sample or as platinum sensitive if the ratio of the second biological sample is less than the ratio of the first biological sample.

In some embodiments, the method disclosed herein further comprises diluting the biological sample (e.g., dilute the biological sample about 1.5 times, 2 times, 5 times, 10 times, 20 times, 50 times, 100 times, 500 times, 1000 times, or 10,000 times) prior to the step of contacting the biological sample with the test paper.

Also disclosed herein is a kit for monitoring recurrence of a cancer (e.g., ovarian cancer, endometrial cancer, or lung cancer) in a subject, wherein the kit comprises a test paper comprising a first reagent area that changes in color shade after contacting a human epididymis protein 4 (HE4) polypeptide and a second reagent area that changes in color shade after contacting a creatinine (CRE) polypeptide. In some embodiments, the test paper comprises the first reagent area or the second reagent area. In some embodiments, the test paper comprises the first reagent area and the second reagent area. The test paper can be used for detecting HE4 and/or CRE by contacting with a biological sample (e.g., biological sample from urine, menstrual blood, or airway fluid).

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Introduction

Human epididymis protein 4 (HE4) is a glycoprotein that is highly expressed by ovarian carcinomas. HE4 in serum is overexpressed in ovarian carcinomas and is detected with high sensitivity and specificity. Recent studies have shown that HE4 in serum offers more forecasting power for OC recurrence than CA-125, a biomarker of many cancers. In 2009, FDA approved using serum HE4 levels to monitor ovarian cancer recurrence (OCR) risk, but this method also requires travel to clinic, phlebotomy, and it is expensive. Due to the ease of collection of urine, HE4 in urine has emerged as a promising target in ovarian cancer recurrence monitoring at-home. Urine HE4 has been found to be a biomarker for ovarian neoplasms with improved sensitivity in early disease compared to HE4 in serum; additionally, HE4 is detectable in urine earlier than in serum. Unlike serum HE4, urine HE4 fluctuates with volume, and creatinine (CRE) is commonly used as an internal standard to normalize the ratio of urinary biomarkers and HE4 in particular. The ratio of HE4/CRE is found to be a better predictor of early and late stage ovarian cancer than urine HE4 alone with highly predictive cut-off range of 3.5. Higher concentrations of HE4/CRE ratios were shown in ovarian cancer patient urine ranging from −0.5 to 2.5 for log 10 values of HE4/CRE. 0.3-1.67 log 10 values were tested, which are within the range of human samples. The initial rising of values of HE4/CRE observed in recurrence was tested.

HE4/CRE can range from 2 in healthy patient samples with to nearly 45 (although most are less) in late stage ovarian cancer. Importantly, Urine HE4 can also be more useful than serum HE4 in differentiating low malignant potential cysts from early ovarian cancer.

Due to their ease of use, low cost, and robust manufacturing, paper-based tests, such as lateral flow assays (LFAs), are attractive tests for both at-home testing and low-resource testing. Lateral flow assays rely on a colorimetric reaction that can be read by the human eye as a simple yes/no result. In these yes/no applications, improving the limits of detection is the major challenge. However, both HE4 and creatinine are present in large quantities and are easily detectable in urine, so a lower threshold is not a concern. In addition to qualitative uses, LFAs have also been used for quantitative detection of various proteins or nucleic acids. The challenge in a paper-based test for HE4/CRE is accurate quantification of HE4 and creatinine values in the clinically relevant ranges. For quantification of LFAs, an optical instrument is required for capturing and measurement of the output. Owing to their portability, low-cost, and widespread use, cell phones are well-studied instruments to quantitate LFAs for home-testing and low-resource setting testing.

This report describes the development of an at-home paper-based device to measure both urinary HE4 and urinary creatinine in one device. A single urine sample is applied to both test strips and analyzed at the same time. The approach is based on standard LFA technology and incorporates a creatinine measurement in a low-cost assay format that can be imaged and quantified using a flatbed scanner as well as a cell phone app. This approach was tested using surrogate samples over the clinically important range of HE4/CRE ratios from 2 to 47.

### Example 2. Results

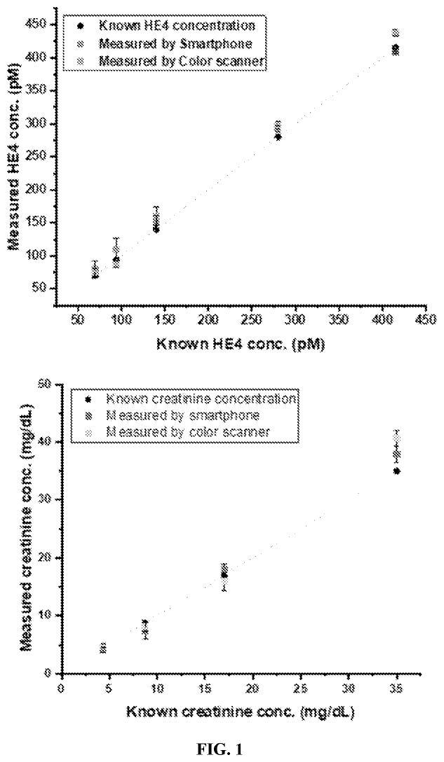

In tests of the accuracy of this approach, the concentration of HE4 or CRE was varied individually (Table 1). Compared to the known sample concentration, the percent error was usually under 16% for both the scanner and cell phone. Scanner vs cell phone performance is compared to the known values of analyte in FIG. 1. To determine the accuracy for one analyte individually, only the HE4 parameter or only the creatinine parameter was varied along the standard curve for each biomarker. A one-way ANOVA was done to compare the actual value, the scanner and the cell phone for both HE4 and CRE values. For HE4, the calculated F value (0.0095) is smaller than our F critical value (3.8853). Similarly, for CRE, the calculated F value (0.0042) is smaller than our F critical value (4.2565). Therefore, the scanner, cell phone and known values are not significantly different for either HE4 or creatinine.

TABLE 1
CRE (n = 3) and HE4 (n = 4) concentrations were calculated from the
standard curve using the flatbed scanner and cell phone and compared
to the actual known value. Percent error for all concentrations is reported.

| Known | Scanner | % | Cell phone | |
|---|---|---|---|---|
| Concentration | Concentration | error | Concentration | % error |
| 4.4 mg/dL CRE | 4.8 mg/dL CRE | 9.9 | 4.2 mg/dL CRE | 4.0 |
| 8.8 mg/dL CRE | 8.2 mg/dL CRE | 6.2 | 7.3 mg/dL CRE | 16.4 |
| 17.5 mg/dL CRE | 15.9 mg/dL CRE | 8.9 | 17.9 mg/dL CRE | 5.8 |
| 35.0 mg/dL CRE | 40.7 mg/dL CRE | 14.1 | 37.9 mg/dL CRE | 8.3 |
| 70 pM HE4 | 66 pM HE4 | 4.8 | 61 pM HE4 | 12.2 |
| 94 pM HE4 | 109 pM HE4 | 16.6 | 102 pM HE4 | 9.2 |
| 140 pM HE4 | 162 pM HE4 | 16.2 | 149 pM HE4 | 7.1 |
| 280 pM HE4 | 287 pM HE4 | 2.5 | 288 pM HE4 | 3.1 |
| 415 pM HE4 | 437 pM HE4 | 5.4 | 425 pM HE4 | 2.6 |

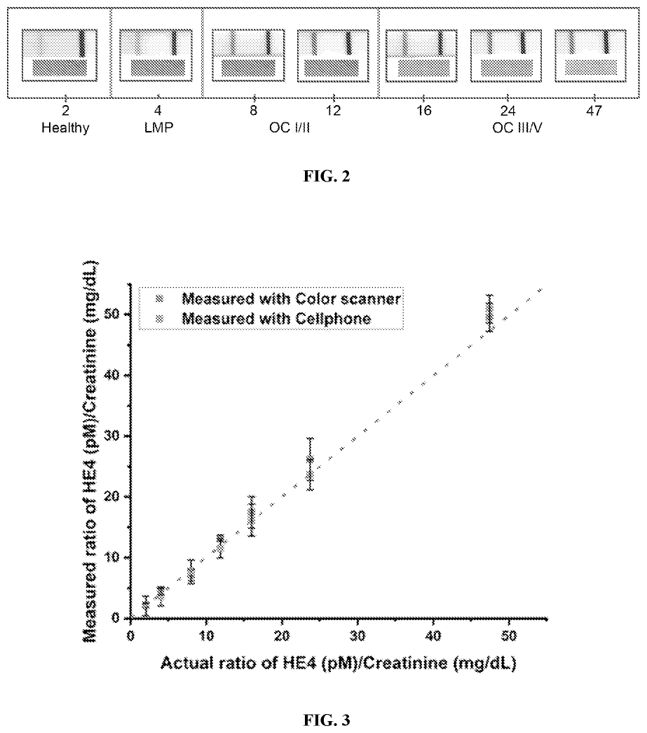

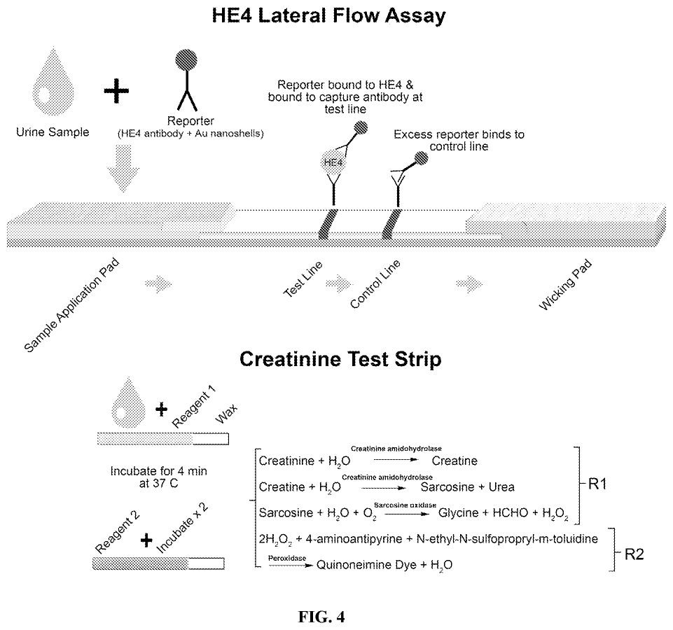

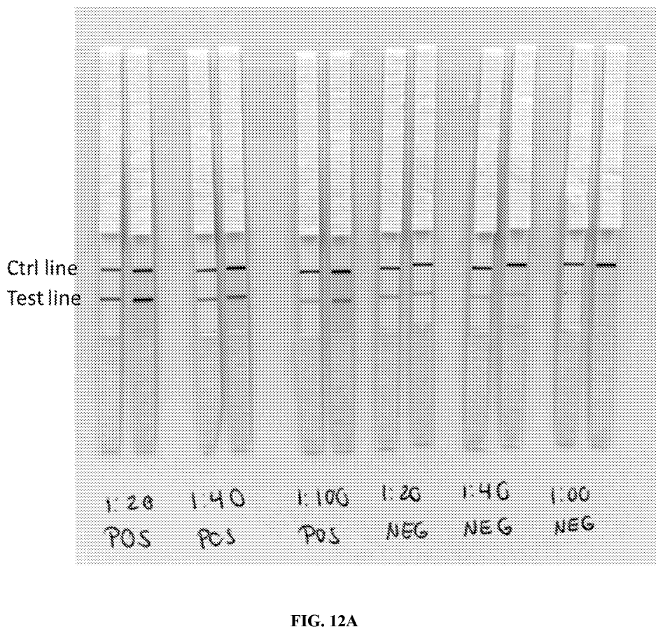

FIG. 2 shows flatbed scanner images covering the range of ratios from 2 to 47. Example cell phone images are contained in FIGS. 11A-11I. As this figure shows one can qualitatively see the ratio has changed by examining the HE4 test line and creatinine test strip color intensity. However, the images show that visual inspection cannot provide a quantitative answer. The quantitative analysis of images (FIG. 2) are compared to the actual ratio of HE4/CRE and the measured ratios by the scanner and cell phone (FIG. 3). A one-way ANOVA test was performed on the matrix values. The F value (0.0148) is smaller than the F critical value (3.4028) so there is no difference among the actual value, the scanner, and the cell phone. The actual values for the known ratio, the scanner ratio and the cell phone with the associated errors are shown in Table 2. The analysis showed that varying creatinine did not significantly alter the intensity of the HE4. Likewise, the level of HE4 did not interfere with the output of the creatinine result.

TABLE 2
Surrogate patient samples created for three HE4 concentrations
across three different creatinine concentrations. Scanner and cell
phone values are reported for each biomarker. Percent error is
reported for each concentration (n = 5). Duplicate ratios are not shown

| Known Ratio | Scanner Ratio | % error | Cell phone ratio | % error |
|---|---|---|---|---|
| 2 | 2.2 | 9.77 | 2.1 | 3.74 |
| 4 | 4.4 | 11.06 | 3.6 | 8.74 |
| 8 | 7.1 | 10.72 | 7.7 | 5.02 |
| 11.9 | 13.2 | 11.33 | 11.5 | 2.67 |
| 16 | 17.4 | 9.04 | 16.2 | 1.08 |
| 23.7 | 23.7 | 10.33 | 23.6 | 0.28 |
| 47.4 | 49.6 | 4.50 | 50.9 | 7.40 |

### Example 3. Discussion

This study shows that the method to measure HE4/CRE with a cell phone or scanner worked well over the clinical ranges for ovarian cancer recurrence. The application for this device is aimed at repeated measurements within the same individual. This ratio changes with stage of ovarian cancer. When applied to a single individual, these ratios can mirror the increases found in group average values. Percentage error for the surrogate testing was usually less than 27% for the scanner, with the average percent error being 10%. The worst percent error for the cell phone was better at 12% and was only 4.13% percent error on average for the cell phone. it is validated with clinical samples. Recurrence was defined by a doubling of the HE4 value in serum. Since urine HE4/CRE correlates positively with serum HE4, this method has much less error than the expected doubling in recurrence. Therefore, if a user sees a doubling in their HE4/CRE ratio, this is well outside the observed error range and should be interpreted as a change in their risk of recurrence.

These results indicate that the cell phone app provides similar results to the flatbed scanner (FIG. 2 & FIG. 3). The particular brand of flatbed scanner was an ideal comparison to the cell phone app since both devices use LED illumination. Additionally, ImageJ was used to measure the intensity of the LFA and creatinine test. The custom phone app automatically analyzes the intensity of the test, which can reduce human error compared with the manual measurement method in ImageJ. The cell phone app also produces results much faster compared to the scanner. The custom cell phone app allows a more streamlined and robust process for users as well as reduced cost and space over that required for a flatbed scanner without a significant difference in results. Additionally, the cell phone app analyzed two tests in a single image. This enables the comparison between HE4 and CRE, but it can be done for any analyte in a sample. This streamlined process, affordability, and comparable accuracy to the scanner indicates that this device shows promise as a valuable platform for home testing.

The HE4/CRE test ranges were chosen based on published reports on the levels found in urine in early recurrence. The values of HE4 concentration are inside the clinical urine ratio ranges. Since the HE4 concentrations in urine are much higher than the LoD of the assay shown herein, the shown method can be used with higher dilution factors. The normal creatinine range is around 20-275 mg/dL in women. Ranges to 4.4 to 35 mg/dL were tested to include the lower ranges of urine based on the need to dilute the urine sample. HE4 concentrations in urine are roughly 10-10,000 pM. Ranges of 28-543 pM were tested based on the assumption of a 1:40 dilution. Since the use of the technology is for recurrence monitoring, where levels can be lower, where the initial rise can occur was tested for greatest impact.

In contrast with previous work, the device shown herein does not require the complicated infrastructure necessary to perform ELISA, making it more adoptable for home-testing. Previous work coupled a microchip ELISA with a cell phone mobile application that can detect the HE4 biomarker in urine from ovarian cancer patients. However, the work did not normalize HE4 to creatinine. Also, an ELISA microplate and ELISA microchip both require numerous sample processing steps (i.e., three washing steps, several hour incubation steps, and manual mixing of the final solution), which increase cost and error. Limited lab infrastructure and skilled technicians are not widely available in low resource settings, which make ELISA and ELISA-based devices difficult to access. The urine LFA shown herein is easily integrated with any cell phone with the customized app. The signal from the test line can be quantified by the custom designed urine LFA, making it possible to detect changes in HE4 level in urine by untrained users. Without the phone, this entire device is under $5.00 to create.

One application for this device is to facilitate biomarker tracking over time with patient-specific baseline HE4/CRE levels. Although many academic papers have demonstrated various devices or methods that can differentiate healthy and OC urine, these devices have not been translatable in a clinical setting. Due to variation of OC (stage, grade or subtype), a single biomarker cut-off value may not very be useful at the individual patient level. However, measuring the amount of HE4 urine when first diagnosed with OC and again after debulking surgery and treatment can provide physicians with two critical biomarker points to compare (disease level and no evidence of disease level). By serially monitoring HE4/CRE in urine and comparing patient-specific levels, users can be presented with new biomarker changes to guide further invasive testing.

This type of testing method is useful in less-developed countries, which face a rapid rise in cancer incidence without increased infrastructure investments in healthcare. Due to this constraint, methods that can detect biomarkers in human samples remotely without the need for training or expensive equipment will have greater adoptability. Low-cost and easy-to-use cancer management strategies can benefit less-developed regions as well as more developed countries. In 2013, the World Health Organization (WHO) endorsed self-sampling as an option for the initial screening for Human Papilloma virus (HPV) for low-to middle-income countries in an effort to reduce cervical cancer death. Studies by Kamath et al showed that self-sampling for cervical cancer screening in low-resource environments was not just convenient, it was also able to reduce disparities in access to screening and reduce mortality.

Another application of this technology is to monitor early therapy responses. Serial measurements of HE4 in urine show that mean urine HE4 levels remained stable (7% decrease) in patients that proved to be platinum resistant, while they decreased 68% in those that proved to be platinum sensitive. This indicates that urine HE4 levels can also be a useful tool for monitoring recurrence in patient with platinum resistance. Urine HE4 is studied further to evaluate the predictability of chemotherapy response. One way to do this is by using the methods described herein. This remote testing method indicates the need to switch to a more effective therapy in a timely manner when survival outcomes are higher.

One of the most impactful use of this technology is in early detection of ovarian cancer. Currently, there is no recommended screening test for ovarian cancer. Since ovarian cancer presents subtly and physicians lack sensitive diagnostic tools, most diagnoses occur in later stages in which treatment options are often limited as well as costly. High-risk individuals (patients with a first-degree relative with ovarian cancer or carrier of a BRCA mutation), often undergo pelvic exams combined with Transvaginal Ultrasound (TVU) and blood test for changes in CA-125 levels even though these strategies have shown no proven benefit for reducing ovarian cancer mortality. One study examined a “2-out-of-3” decision rule that required 2 of 3 serum biomarker tests to be positive. Application of this rule identified 100% of the high-risk cases and showed a false-positive rate of 1.5%. This indicates that multiple positive tests can help identify high risk patients for more follow-up testing at clinics. Longitudinal measurements of HE4 within a single patient with a genetic risk can be useful as an early detection tool for gynecological oncologist. In this use, if women who are at genetic risk can record their HE4 levels 3-4 times a year, a significant rise in HE4 can be used to suggest follow-up screening. Results help guide decisions on the timing of more invasive procedures such as tissue biopsy or prophylactic ovary removal.

The current technology is a good match for lateral flow testing and the high abundance of analytes of interest in the urine. As both HE4 and creatinine are present in high amounts and much higher than the limit of detections in lateral flow tests and dilutions of urine is necessary prior to application to test strip. A dilution step presents an additional step for a patient and is more error prone as a result. In some embodiments, to offset this, a specific-sized pipette coupled with a pre-aliquoted deionized water vial is combined easily to help offset errors in measurement. In fact, many pregnancy tests require users to use the included small pipette and deliver only 3-4 drops onto the sample pad. As many women are well accustomed to using such tests, this indicates a pre-aliquoted water vial can be added to mix the pipetted. A step-by-step guide with pictures or a “how to” video on the cell phone app can assist users in proper test procedure.

In addition, most commercial LFAs include a spray-dried conjugate release pad in their test format. The devices and methods in the examples herein did not use an air jet dispenser to spray our reporter onto a release pad. Instead, the sample was combined with the reporter in a single Eppendorf tube for 10 minutes and spotted onto the sample application pad.

### Example 4. Methods

Reagents. Monoclonal mouse anti-human epididymis protein 4 (HE4) was purchased from HyTest (Turku, Finland). The reporter reagent used in the LFA was BioReady 150 nm gold nanoshells by nanoComposix Inc (San Diego, California). Ethyl-N;-(3-dimethylaminopropyl) carbodiimine hydrochloride (EDC) and Sulfo-NHS chemistry was used (sigma Cat #E1769-1G, Thermo, Prod #24510) to covalently bind the antibody to the nanoshell. Hydroxylamine (Sigma, cat #467804) was used to quench remaining amine groups during the covalent conjugation protocol.

HE4 antigen standards were provided by Fujirebio Diagnostics Inc. (Malvern, PA). For the enzymatic creatinine reaction, a Creatinine LiquiColor kit was purchased from Stanbio (Boerne, TX). The creatine test paper used was Whatman no. 3, purchased from Cytiva (Marlborough, MA). The dipping soy wax was purchased from Hearts & Crafts (Brooklyn, NY). Parafilm tape was purchased from Hach (Loveland, CO). Anhydrous creatinine was purchased from Thermo (Prod #C4225, lot #SLCF5841). All solutions were prepared with deionized water unless otherwise noted.

Lateral Flow Materials & Design. To create the lateral flow assay standards for HE4, lyophilized standards were used in the Fujirebio ELISA kit, which contained HE4 antigen in a phosphate buffered salt solution with bovine serum albumin, an inert yellow dye, and a non-azide antimicrobial preservative. To reconstitute, 1.0 mL of deionized water was added to each standard vial. The vial was vortexed and then allowed to stand at room temperature for 15 minutes. Prior to use, the vial contents were gently mixed by pipette. Standard dilutions were created from the standards for the simulated patient samples with creatinine solutions or deionized water. Vials were stored at 4° C. before and after use as recommended by the manufacturer.

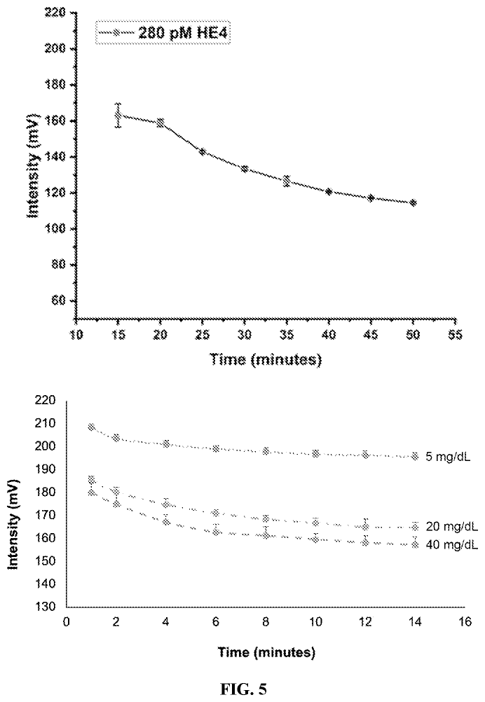

Both HE4 and creatinine were measured using paper-based methods (FIG. 4). For HE4, standard LFA methods were used to spot reagents and assemble the test strips. An automatic antibody dispenser (IsoFlow from Imagene Technology Inc) was used to deposit HE4 sheep-derived polyclonal antibody (R&D Systems, Cat #AF6274) across the membrane as the capture antibody at the test line. To make the control line, the antibody dispenser deposited a goat anti-mouse antibody (Lampire, cat #7455507, Lot #17H40070) across the membrane. The mouse antibody was chosen for the control line so that the excess mouse antibody can bind to it. The striped membrane was dried for one hour in an oven at 37° C. Next, the striped membrane was placed onto an adhesive backing card. A sample pad was added to the right edge of the membrane and the wicking pad was added to the left edge, overlapping the membrane by 1 mm to allow for capillary action along the test strip. The assembled card was cut into individual test strips 3.9-mm wide by 70-mm long with an automated guillotine (Matrix 1201 Membrane cutter, Kinematic, Twain hart, CA). The strips were stored at room temperature in sealed pouches with desiccants until use.

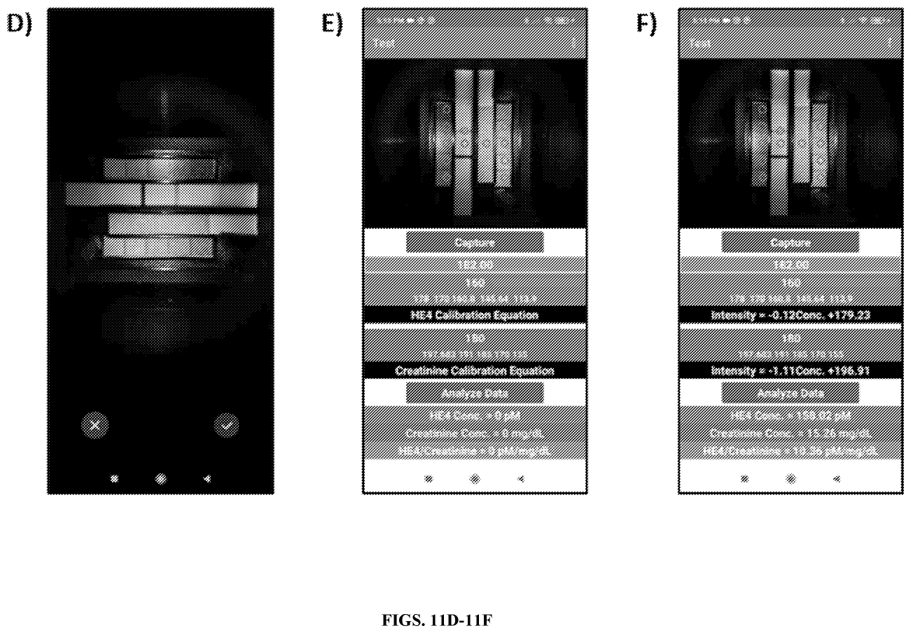

Next, the selection of the reporter antibody and nitrocellulose membranes were optimized. To identify the best reporter, three monoclonal HE4 reporter antibodies (R&D systems, LsBio, and Hytest) were screened. To test the binding and non-specific binding of each antibody a dot blot test was conducted. 1 μl spot of polyclonal antibody was spotted on the membranes and the membrane was dried for 1 hour in an oven at 37° C. 1 μl of antigen was then mixed with each of the reporters and the solution was spotted onto the sample pad (FIG. 9) for the positive control test. For the negative control test, the reporter was spotted without antigen. Non-specific binding occurred when a colored spot was generated without antigen present. Unbound reporters were washed away by the addition of a running buffer to the sample pad. Since the Hytest antibody showed strong signal intensity in antigen binding in the positive control tests and the least non-specific binding in the negative control tests, it was chosen for further testing.

Three different nitrocellulose membranes were screened (FIG. 9) and MDI CNPC (10 μm) membranes (MDI membrane technologies) were subsequently chosen since they exhibited the least non-specific binding in the dot blot test, and the antigen signal intensity was higher in the positive control tests.

To run the LFA HE4 test, 20 μl of nanoshell+HE4 antibody was used incubated with 25 μl of sample for 10 minutes on a rotator prior to pipetting it onto the sample pad of the lateral flow test strip. After deposition onto the sample pad, the lateral flow test was left at room temperature before measurement on a flatbed scanner or cell phone.

Creatinine Test Design. For the design and fabrication of the creatinine test, an enzymatic creatinine kit was used that comprises a two-reagent system that is typically found in automated analysis equipment. Since chemicals can interfere with creatinine determination using the Jaffe reaction, enzymatic reagents were used to measure creatinine. Enzymatic reaction has been chosen over the picrate reaction for the determination of creatinine in clinical labs as the results of enzymatic methods have been reported to match the gold standard method more closely, isotope-dilution-mass spectrometry (IDMS). In the first reaction mixture, reagent 1 (R1), the creatinine amidohydrolase, converts creatine to sarcosine, and the oxidation of sarcosine by sarcosine oxidase produces hydrogen peroxide. The hydrogen peroxide generated in R1 is then reacted with reagent 2 (R2) in the presence of peroxidase to react with Quinoneimine dye as shown in FIG. 4. This reaction results in a light to dark purple dye in which the dark purple corresponds to high creatinine concentration in the sample.

Whatman no. 3 paper was used for its high absorbency, medium porosity and medium flow rate. To create the creatinine paper strips, Whatman no. 3 paper was cut with scissors into individual 2.5-mm long by 10-mm wide pieces. The test strip end was then briefly dipped in heated liquid soy wax. The wax end created a barrier for the chemical products generated during the chemical reaction and allowed for tweezer handling of the test strips.

As powdered creatinine dissolves readily in deionized water, a stock solution of creatinine at 80 mg/dL was created. Standard dilutions were made from the stock solution with deionized water into individual Eppendorf tubes and refrigerated at 4° C. until use. The creatinine enzymatic kit uses a 300 μl cuvette to measure absorbance and the manufacturer instructions suggest using 270 μl of R1 and 90 μl of R2. For our small paper-based tests, we reduced the volumes for the two creatinine enzymatic reagents (R1 and R2) but preserved the ratio as suggested by the manufacturer. This reduction in volume was necessary to prevent leaching out of the paper onto the cell phone housing or scanner bed during measurement. We pipetted 18 μl of R1 onto the test strip followed by 6 μl of sample. The test strip was then placed in a 37° C. oven for 4 min. Then the test strip was removed from the oven, and 6 μl of R2 was pipetted on the test strip. The test strip was then placed back into the 37° C. oven for 4 min. The incubation in the oven was used to decrease the time to measurement: if left at room temperature, the result can be read in 30 minutes. The test strip was finally removed from the oven and allowed to sit at room temperature before measurement on the flatbed scanner and cell phone. To prevent leaching out, a small 3 mm×11 mm section of Parafilm tape (Hach, Loveland, CO) was added to the phone cassette well where the creatinine test is placed. The hydrophobicity of Parafilm tape ensures that the liquid remains inside the creatinine test strip during measurement.

Quantification of paper-based tests. The quantitative LFA method involves measuring the amount of an analyte in a sample against a standard curve of known amounts of the analyte. To measure the intensity of the reaction in both the LFA and creatinine tests, a CanoScan LIDE 300 scanner was used from Canon (Ota, Tokyo, Japan) and a cell phone (Samsung galaxy s20).

In this report, quantification was compare using the cell phone app to a flatbed scanner as the “gold standard.” Flatbed scanners have been used widely to quantitate LFAs and other colorimetric tests. For the scanner measurement of HE4, the individual paper strips were placed face down. The auto scan button was pressed to create a PDF scan. The document was then opened in Adobe Photoshop to convert the PDF to JPEG. The JPEG was then opened in ImageJ. The test line in the image was measured with the “Measure tool”. The output of the measured result was then copied and pasted into Excel for analysis.

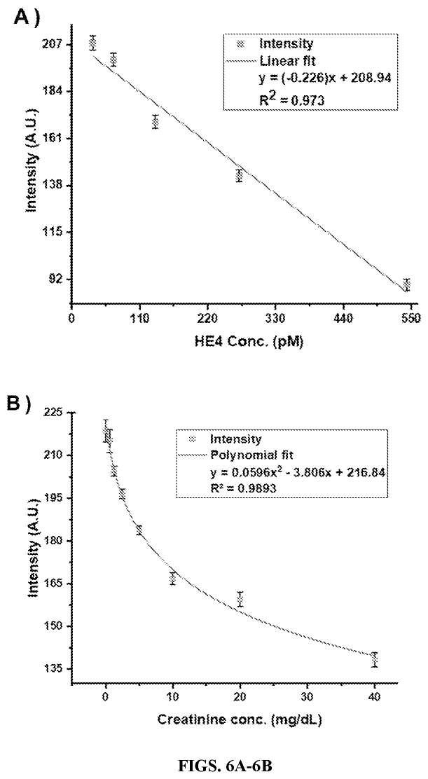

In this study, determination of HE4 concentration was based on a sandwich assay format in a lateral flow method. Therefore, the timing of when the reaction is considered “complete” and ready to read on a scanner or cell phone app influences the intensity values. Per the manufacturer's instructions for the membrane of choice, MDI-10, the strip can be read at 15 minutes after sample application. Therefore, the intensity associated with a concentration of 280 pM (the medium standard) was analyzed over 5-minute intervals from the earliest it could be read (FIG. 5). The 20-minute time mark was chosen because subsequent drying of the test reduced the intensity of the test value by about 12% as shown in FIG. 5.

To determine the optimal time for reading the creatinine test strips, three concentrations of a standard curve were analyzed every two minutes until 14 minutes. The time point at 8-10 minutes was selected as the change from 8-16 minutes is less than 2% change (FIG. 5).

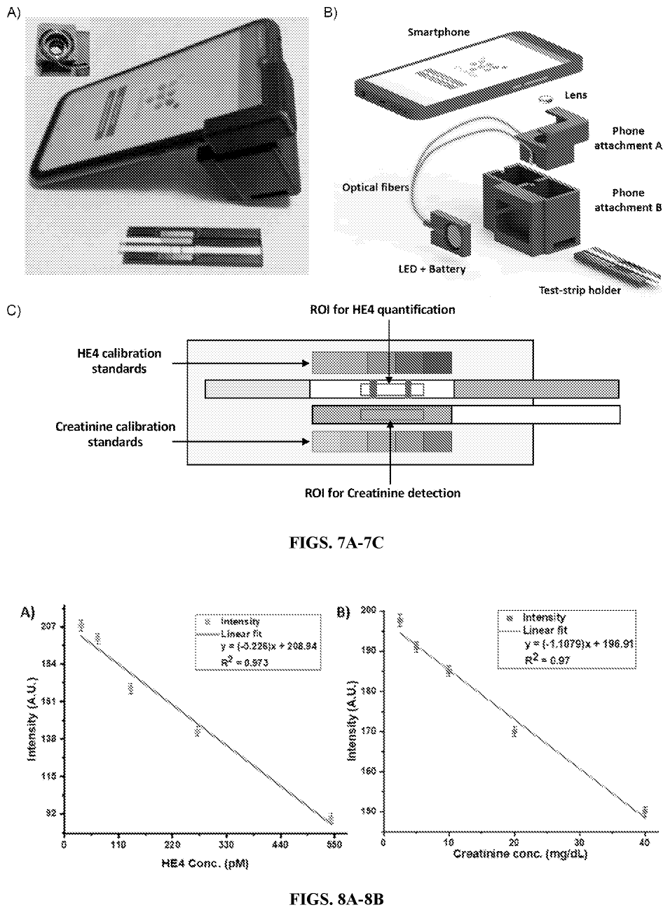

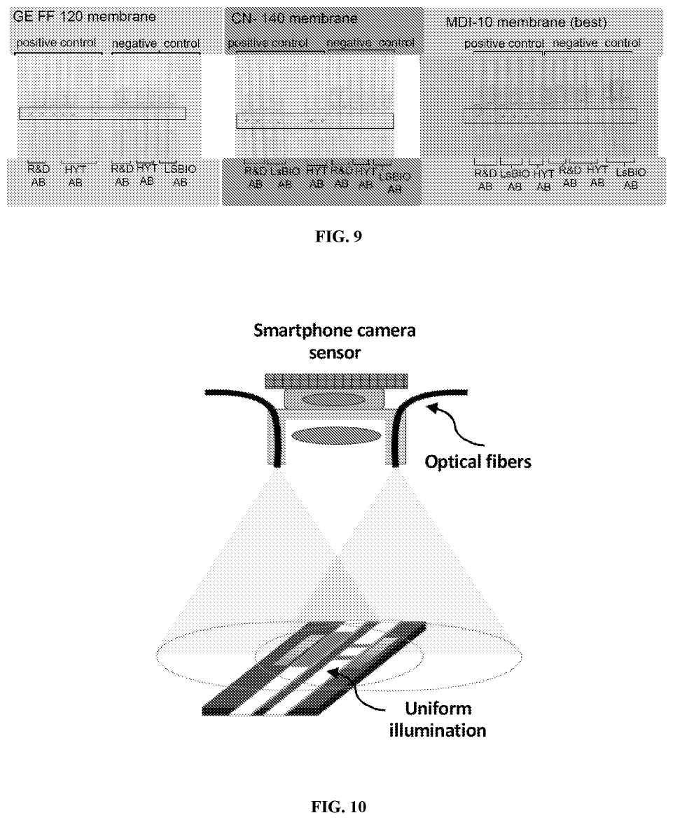

To make a standard curve for HE4, the 573 pM HE4 standard from the ELISA kit was serially diluted. The output of the value calculated from the standard curve was then compared to the known value (FIG. 6A). For the scanner measurement of Creatinine, an 80 mg/dL stock solution of creatinine was made and serially diluted to create a standard curve for creatinine. After incubation, the test strips were allowed to sit at room temperature for 8-10 minutes before being measured in the scanner. Creatinine concentrations up to 40 mg/dL were analyzed, and triplicate assays were performed for each concentration. The results demonstrated a second order polynomial fit range from 0 mg/dL to 40 mg/dL of creatinine (r²=0.9893), as shown in FIG. 6B. The limit of detection (LOD) of each test strip was determined by analyzing a blank 5 times and a 3 SD limit was calculated. The LOD for HE4 is 15 pM and for creatinine test the LOD is 1.2 mg/dL. Average intensity of the blank for is 226.4856+2.9178 for HE4 and 243.31+2.99 for creatinine (FIG. 2). In addition to quantification with a flatbed scanner, a cell phone-based system was also developed for quantification of both HE4 and creatinine. FIG. 7A shows a photograph of the developed device, which includes a 3D-printed black enclosure and a test strip holder. The black enclosure is used to mitigate the problem with varying external lighting conditions. The current system was implemented on an Android platform-based cell phone (Redmi Note 7 from Xiaomi, camera resolution 4000 pixels×3000 pixels), but the same system can be used with other cell phones with a small adjustment in the design of the phone attachment. As shown in FIG. 7B, an inexpensive plano-convex lens of focal length 75 mm was used to magnify the area of the test strips. The lens was attached to the phone attachment A, which is mechanically connected to the cell phone. Phone attachment B is connected to attachment A. The attachments are positioned perpendicular to the phone to eliminate the need to tilt the phone and to standardize the focal distance to reduce imaging inconsistencies. Both attachments are 3D-printed with black polylactic acid (PLA) material. The material of choice is not significant. Black was chosen to decrease the reflectance of light inside the test strip compartment. Once the test-strip holder is positioned inside of Phone attachment B, ambient light is blocked from entering the inside of the phone attachment B.

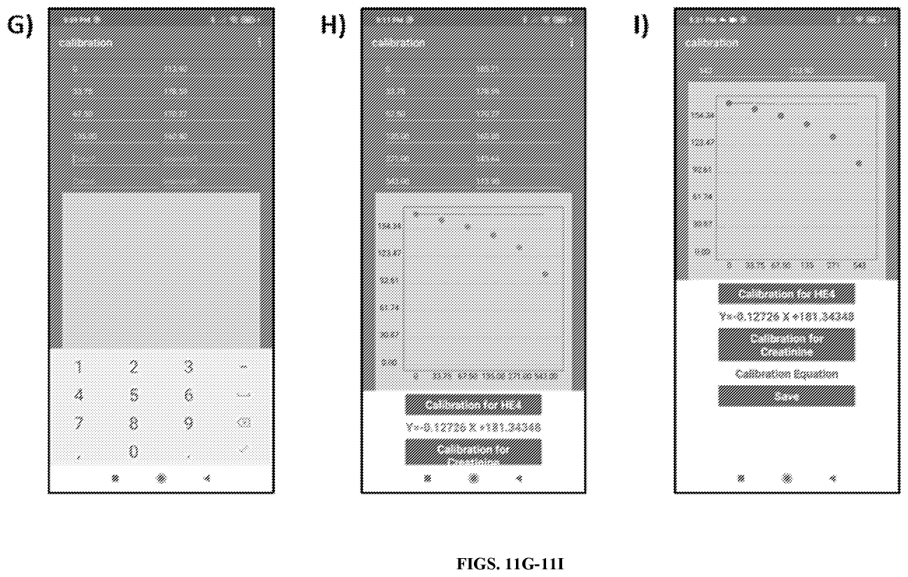

A cell phone flashlight can be used as a light source for colorimetric applications. However, in the current application, it is not possible to use the flashlight to illuminate the test strips. This is attributed due to the use of the external lens to acquire an image with a large field of view to cover both the test strips and the calibration standards. Another problem in using the flashlight is that its position varies from phone to phone, therefore the illumination over the detection area will be different for different brands of cell phones 35. Instead, here uniform illumination was achieved by using a novel optical fiber-based illumination scheme as shown in FIG. 10. Light from a low-cost external battery-powered white LED (Finware LED, Amazon) is coupled to two plastic multimode optical fibers (980-μm core diameter, Edmund Optics), and the other ends of the optical fibers are connected to the phone attachment. The distances between the test strips and the optical fibers were optimized to achieve a uniform illumination area. The battery and the LED compartment are located on the outside of the attachment so that they are easily operable by the user and the battery. While the cell phone battery can be used to power an external LED but long term use of cell phone battery for powering external components heats up the phone and the output power of the LED is significantly reduced when the cell phone battery power is low (<30%). Therefore, in the current application, an external coin cell battery was used to power the LED, which can easily be replaced when needed.

FIG. 7C shows the schematic of the test-strip holder. The test strips are inserted from the side and manually slid into the field of view. The test-strip holder contains the HE4 test, CRE test and calibration stickers to allow for simultaneously measurement of both tests and calibration stickers. Calibration stickers were prepared indicating the color of both the HE4 and CRE standard solutions and attached them to the test-strip holder. The phone application captures both the tested strips and the calibration stickers together in a single image frame and evaluates the intensity values. The intensity values from the calibration stickers are used to generate the respective calibration equations. After that, the unknown HE4 and creatine concentrations are evaluated by interpolating corresponding intensities values in respective calibration equations. This device evaluates the intensity of both tests and determines the corresponding concentration value by using the calibration sticker as a reference standard. The calibration standards are printed as a vinyl sticker to increase stability over time compared to typical inkjet printer paper. To facilitate on-board data processing, an Android application (app) was developed and installed on the cell phone. The detailed procedures for using this app are given in the supplementary information. To simulate an at-home user, both HE4 and creatinine were measured simultaneously on both the scanner and cell phone imaging modality (FIG. 3).

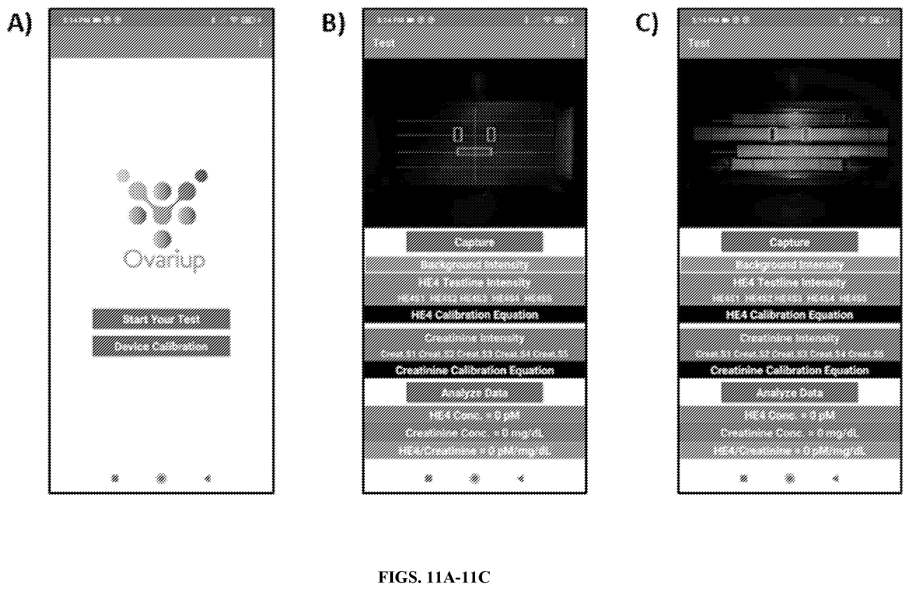

FIG. 6 shows the calibration plots generated from the HE4 (pM) and CRE (mg/dL) standard solutions using the cell phone app. The analysis algorithm in the phone is simple, as the concentration can be determined by a linear relationship. In the case of HE4, the concentration range of 33.75 pM-543 PM has been selected, which indicates a co-efficient of determination (R2) of 0.973 as shown by FIG. 8A. This range was within the range of the ELISA kit. Similarly, for CRE, a concentration range of 2.5 mg/dL-40 mg/dL was evaluated, which shows a co-efficient of determination (R²) of 0.97 as shown in FIG. 8B. For the phone, a linear fit was chosen over a polynomial fit since the linear fit provided a high R² value and was computationally easier since there is only one answer given from the intensity calculation.

Cell Phone app operation. The cell phone app was developed using MIT app inventor 2: a cloud based open-source platform. This app is compatible with any cell phone with an operating system of greater than 2.3 (Gingerbread). The workflow of the app is shown in FIG. 11. FIG. 11A shows the main window of the app, it provides two buttons to the user “Start Your Test” and “Device Calibration”. On clicking the “Start Your Test” button, it will open the test window which contains a live camera view with floating positioning markers as shown in FIG. 11B. The user needs to position the test-strip holder in the device so that the HE4 test line and creatinine test-strip reaction areas are within the boundary of the rectangular markers on the cell phone screen as shown in FIG. 11C. Now, on clicking the “Capture” button, the app pop-ups the in-built phone camera and capture an image of the test strips as shown in FIG. 11D. After capturing the image the app will process the image internally and calculate the grayscale intensity of the HE4 test line, the white area of the HE4 test for reference, creatinine test strip and the intensity of the calibrations stickers marked by the circles. As shown in FIG. 11E, the app changes the place holders texts for the intensity values to the grayscale intensity respectively. On clicking the “Analyze Data” button, the application generates the calibration equation from the intensity values of HE4 and creatinine standards as shown in FIG. 11F and determines the unknown concentration by fitting the intensity values of the biomarkers. Although, the app is self-content to calibration from the calibration stickers but there are situations where a device calibration in needed with freshly prepared standard solutions. To facilitate on-site calibration, the user needs to click on the “Device Calibration” button on the home screen, which opens the calibration window as shown in FIG. 11G. In this window, the user types the standard concentration values for HE4 and the corresponding intensity values into the respective boxes. After inserting the values, the user clicks the “Calibration for HE4” button, the app performs the least square fitting and generates the calibration equation as shown in FIG. 11H. After that on clicking the “Save” button shown in FIG. 11I, the calibration curve can be implemented to the app data processing algorithms. The same procedure can be used to calibrate the device with creatine standards. A copy of the code is provided by request.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of monitoring recurrence of a cancer in a subject, comprising
obtaining a biological sample from the subject's urine;
contacting the biological sample with a first test paper comprising a first reagent area that changes in color shade after contacting a human epididymis protein 4 (HE4) polypeptide and a second test paper comprising a second reagent area that changes in color shade after contacting a creatinine (CRE) polypeptide;
scanning the first test paper and second test paper to determine the intensity of the color shade of the first reagent area and the intensity of the color shade of the second reagent area;
calculating the ratio of the intensity of the color shade of the first reagent area and the intensity of the color shade of the second reagent area; and
determining that the subject has recurrence of the cancer if the ratio is greater than about 2.

2. The method of claim 1, wherein the test paper is scanned by a cell phone or a scanner.

3. The method of claim 1, wherein the cancer is ovarian cancer.

4. The method of claim 1, further comprising administering to the subject a therapeutically effective amount of a cancer therapeutic agent if the subject has recurrence of the cancer

5. The method of claim 4, wherein the cancer therapeutic agent is selected from the group consisting of cisplatin, carboplatin, paclitaxel, and docetaxel.

6. A method of treating a cancer in a subject in need thereof, comprising
obtaining a biological sample from the subject's urine;
contacting the biological sample with a first test paper comprising a first reagent area that changes in color shade after contacting a human epididymis protein 4 (HE4) polypeptide and a second test paper comprising a second reagent area that changes in color shade after contacting a creatinine (CRE) polypeptide;
scanning the first test paper and second test paper to determine the intensity of the color shade of the first reagent area and the intensity of the color shade of the second reagent area;
calculating the ratio of the intensity of the color shade of the first reagent area and the intensity of the color shade of the second reagent area;
determining that the subject has recurrence of the cancer if the ratio is greater than about 2; and
administering to the subject a therapeutically effective amount of a cancer therapeutic agent if the subject has recurrence of the cancer.

7. The method of claim 6, wherein the test paper is scanned by a cell phone or a scanner.

8. The method of claim 6, wherein the cancer is ovarian cancer.

9. A method of monitoring recurrence of a cancer in a subject, comprising
a) obtaining a first biological sample from the subject's urine immediately after a cancer therapy;
b) obtaining a second biological sample from the subject's urine after step a);
c) contacting the first biological sample and the second biological sample with a first test paper, wherein the first test paper comprises a first reagent area that changes in color shade after contacting a human epididymis protein 4 (HE4) polypeptide and a second test paper comprising a second reagent area that changes in color shade after contacting a creatinine (CRE) polypeptide;
d) scanning the first test paper and second test paper to determine the intensity of the color shade of the first reagent area and the intensity of the color shade of the second reagent area;
e) calculating the ratio of the intensity of the color shade of the first reagent area and the intensity of the color shade of the second reagent area for the first biological sample and the second biological sample respectively, and
f) determining that the subject has recurrence of the cancer if the ratio of the second biological sample is greater than the ratio of the first biological sample.

10. The method of claim 9, wherein the test paper is scanned by a cell phone or a scanner.

11. The method of claim 9, wherein the cancer is ovarian cancer.

12. The method of claim 9, wherein the cancer therapy is a surgery or a chemotherapy treatment.

13. The method of claim 12, wherein the cancer therapy partially or fully eliminated the cancer in the subject.

14. The method of claim 9, further comprising administering to the subject a therapeutically effective amount of a cancer therapeutic agent if the subject has recurrence of the cancer.

15. The method of claim 14, wherein the cancer therapeutic agent is selected from the group consisting of cisplatin, carboplatin, paclitaxel, and docetaxel.

16. A method of determining a subject's response to a cancer therapy, comprising
obtaining a first biological sample from the subject's urine prior to the cancer therapy;
obtaining a second biological sample from the subject's urine after the cancer therapy;
contacting the first biological sample and the second biological sample with a first test paper, wherein the first test paper comprises a first reagent area that changes in color shade after contacting a human epididymis protein 4 (HE4) polypeptide and a second test paper comprising a second reagent area that changes in color shade after contacting a creatinine (CRE) polypeptide;
scanning the first test paper and the second test paper to determine the intensity of the color shade of the first reagent area and the intensity of the color shade of the second reagent area;
calculating the ratio of the intensity of the color shade of the first reagent area and the intensity of the color shade of the second reagent area for the first biological sample and the second biological sample respectively, and
determining:
the subject as having resistance to the cancer therapy if the ratio of the second biological sample is equal or greater than the ratio of the first biological sample; or
the subject as responding to the cancer therapy if the ratio of the second biological sample is less than the ratio of the first biological sample.

17. The method of claim 16, wherein the test paper is scanned by a cell phone or a scanner.

18. The method of claim 16, wherein the cancer is ovarian cancer.

19. The method of claim 16, wherein the cancer therapy is a surgery or a chemotherapy treatment.

20. The method of claim 19, wherein the cancer therapy partially or fully eliminated the cancer in the subject.

21. The method of claim 16, further comprising administering to the subject a therapeutically effective amount of a cancer therapeutic agent if the subject has recurrence of the cancer.

22. The method of claim 21, wherein the cancer therapeutic agent is selected from the group consisting of cisplatin, carboplatin, paclitaxel, and docetaxel.
